(19) United States
(12) Patent Application Publication
(10) Pub. No.: US 2024/0268949 A1
(43) Pub. Date: Aug. 15, 2024

(54) INTERNAL BRACE DEVICES AND METHODS

(71) Applicant: Christian N. Anderson, Nashville, TN (US)

(72) Inventor: Christian N. Anderson, Nashville, TN (US)

(21) Appl. No.: 18/439,594

(22) Filed: Feb. 12, 2024

Related U.S. Application Data
(60) Provisional application No. 63/444,978, filed on Feb. 12, 2023.
(60) Provisional application No. 63/444,976, filed on Feb. 12, 2023.

(51) Int. Cl. A61F 2/08; A61B 17/04

(52) U.S. Cl. CPC A61F 2/0811; A61B 17/0401; A61B 17/0466; A61B 2017/0404; A61B 2017/0414; A61B 2017/0458; A61B 2017/0459; A61B 2017/0464; A61F 2002/0823; A61F 2002/0852; A61F 2002/0858; A61F 2002/0882

(57) ABSTRACT

An internal brace device includes a fixed loop and first and second strands extending from the fixed loop. Each strand forms an adjustable loop, reverses back toward the fixed loop, and passes through a self-cinching section wherein tag ends of the first and second strands may be pulled in a direction away from the adjustable loops to tighten the adjustable loops. A first anchor may be disposed on the fixed loop, and a second anchor may be disposed on the adjustable loops or on a free loop disposed on the adjustable loops. The internal brace is configured to be tightened between the first and second anchors for mechanically bracing a joint. Each anchor may include a rigid anchor or a soft anchor in various embodiments.

### CROSS-REFERENCE TO RELATED APPLICATIONS

This application claims benefit of and priority to U.S. provisional application 63/444,978 filed Feb. 12, 2023 entitled Static Internal Brace Devices and Methods, all of which is hereby incorporated by reference in its entirety. This application also claims benefit of and priority to U.S. provisional application 63/444,976 filed Feb. 12, 2023 entitled Suture Button and Methods, all of which is hereby incorporated by reference in its entirety.

### TECHNICAL FIELD

The present disclosure relates to devices and methods for orthopedic surgery, and more particularly to devices and methods for stabilizing a joint using flexible suture devices and methods to repair and reconstruct ligaments and tissue.

### BACKGROUND

Conventional devices and methods for repair and reconstruction of ligaments and tissue include flexible sutures that may be attached to tissue and anchored to surrounding tissue or bone to place the tissue in an anatomically correct position to facilitate healing and growth. Conventional suture constructs are generally secured to tissue by forming knots in the suture material to maintain tension on the tissue; however, such knots can be difficult to employ in the surgical field, and the knots may loosen over time which leads to slow healing of the tissue.

Further, conventional devices and methods for performing repair of the anterior cruciate ligament (ACL) involve securing the damaged ligament to a suture device and fixating the ligament toward the femur using a suture and an anchor. However, such conventional devices and methods place significant strain on the damaged ligament and do not securely brace the joint to transfer stresses away from the ligament during healing, which prolongs the time needed to heal and may lead to future injury of the ligament.

Additionally, conventional knotted devices have drawbacks in specific procedures where a tendon must be drawn into a narrow socket, tunnel or near a bone with a limited range of motion. Conventional suture devices have a tendency to bottom out when a travel limit is reached, which may limit the amount of tension that can be applied to the device.

What is needed then, are improvements in devices and methods of repairing and reconstructing tissue in orthopedic procedures, and specifically during ACL repair.

### BRIEF SUMMARY

The present disclosure provides devices and methods of use including flexible suture materials that may be attached to tissue such as, but not limited to, the anterior cruciate ligament (ACL), posterior cruciate ligament (PCL), medial collateral ligament (MCL), medial or lateral meniscus, biceps tendon, quadriceps tendon, or other anatomical features in the various joints in humans and animals for repair or reconstruction during orthopedic surgery.

In some embodiments, the present disclosure includes an internal brace device and associated methods for performing knee surgery, such as but not limited to repair or reconstruction of the ACL, including one or more adjustable loops having a self-cinching feature allowing tension to be applied and maintained against the tissue by a self-cinching mechanical engagement of the suture with itself or with a second suture strand. The self-cinching features of the devices disclosed herein may provide numerous advantages over conventional devices and methods that rely on knots tied in the suture material after manual tension is applied, or which disregard the use of an internal brace altogether.

In further embodiments, the present disclosure provides static internal brace devices and methods that may be used to perform ACL repair surgery using a knotless, self-cinching suture construct. The internal brace suture device and methods offload stress from the repaired ligament to allow rehabilitation without excessive stress applied to the repaired tissue. In some embodiments, the internal brace holds tension on the knee joint while a separate suture construct engages the ligament to be repaired and pulls the ligament toward the correct anatomical position.

In further embodiments, the present disclosure provides internal brace devices and methods employing the use of one or more soft anchors to secure the internal brace to the femoral and tibial sides of the knee joint. The use of soft anchors provide advantages over conventional rigid anchors because the soft anchors are able to conform precisely to the unique contour of the bone or tissue at the bone tunnel orifice. Additionally the soft anchor material may compress against the bone or tissue without sacrificing tension, and may provide a more comfortable fixation device for the patient.

Numerous other objects, advantages and features of the present disclosure will be readily apparent to those of skill in the art upon a review of the drawings and description as set forth below.

### DESCRIPTION OF THE DRAWINGS

FIG. 1 illustrates an embodiment of an internal brace including first and second adjustable loops and first and second self-cinching sections.

FIG. 2 illustrates an alternative embodiment of an internal brace including first and second adjustable loops and first and second self-cinching sections with a free loop on the first and second adjustable loops.

FIG. 3 illustrates an alternative embodiment of an internal brace including first and second adjustable loops and first and second self-cinching sections with an anchor on a fixed loop and an anchor on the first and second adjustable loops.

FIG. 4 illustrates an alternative embodiment of an internal brace including first and second interlocking adjustable loops and first and second self-cinching sections.

FIG. 5 illustrates an alternative embodiment of an internal brace including first and second independent adjustable loops and first and second self-cinching sections.

FIG. 6 illustrates an alternative embodiment of an internal brace including first and second adjustable loops and first and second self-cinching sections and a free loop on the adjustable loops with shuttling sutures on each end.

FIG. 7 illustrates an alternative embodiment of an internal brace including first and second adjustable loops and first and second self-cinching sections with an anchor on a fixed loop and shuttling sutures on each end.

FIG. 8 illustrates an alternative embodiment of an internal brace including first and second adjustable loops and first and second self-cinching sections with anchors on a fixed loop and on the first and second adjustable loops and shuttling sutures on each end.

FIG. 9 illustrates an alternative embodiment of an internal brace including first and second adjustable loops and first and second self-cinching sections with anchors on a fixed loop and on a free loop on the first and second adjustable loops and shuttling sutures on each end.

FIG. 10 illustrates an embodiment of an internal brace inserted in a fixation device.

FIG. 11 illustrates a method of securing a free loop on an internal brace onto a fixation device using a hitch.

FIG. 12 illustrates an alterative embodiment of an internal brace with a free loop secured to both the adjustable loops and a fixation device.

FIG. 13 illustrates an embodiment of a method of positioning an internal brace for installation in a knee.

FIG. 14 illustrates an embodiment of a method of positioning an internal brace for tensioning in a knee.

FIG. 15 illustrates an embodiment of a method of tensioning an internal brace.

FIG. 16 illustrates an embodiment of a method of tensioning an internal brace.

FIG. 17 illustrates an alternative embodiment of an internal brace positioned for tensioning in a knee.

FIG. 18 illustrates an alterative embodiment of an internal brace positioned for tensioning in a knee.

FIG. 19 illustrates an embodiment of an internal brace assembly including an internal brace suture construct and a ligament fixation suture construct.

FIG. 20 illustrates an embodiment of an internal brace assembly including an internal brace suture construct and a ligament fixation suture construct.

FIG. 21A-G illustrate alternative configurations and methods of forming self-cinching sections of an internal brace.

FIG. 22 illustrates an embodiment of an anchor for use in an internal brace.

FIG. 23 illustrates an embodiment of an anchor for use in an internal brace.

FIG. 24 illustrates an embodiment of an anchor for use in an internal brace.

FIG. 25 illustrates an embodiment of an anchor for use in an internal brace.

FIG. 26 illustrates an embodiment of an anchor for use in an internal brace.

FIG. 27 illustrates an embodiment of an anchor for use in an internal brace.

FIG. 28 illustrates an embodiment of an anchor for use in an internal brace.

FIG. 29 illustrates an embodiment of an anchor for use in an internal brace.

FIG. 30 illustrates an embodiment of an anchor for use in an internal brace.

### DETAILED DESCRIPTION

The present disclosure provides a knotless self-cinching static internal brace device and methods for use during orthopedic surgery. The device in some embodiments is configured to be shuttled across the knee from the femur to the tibia, or vice versa, and fixed on each end with an anchor. The internal brace device acts to offload stress from the repaired ligament to allow rehabilitation without excessive stress applied to the repaired tissue. A separate suture construct may be used in addition to the internal brace to secure the ligament to other bone or tissue in an anatomically correct position to facilitate healing of the ligament, without requiring the ligament to bear the complete load incurred by the joint.

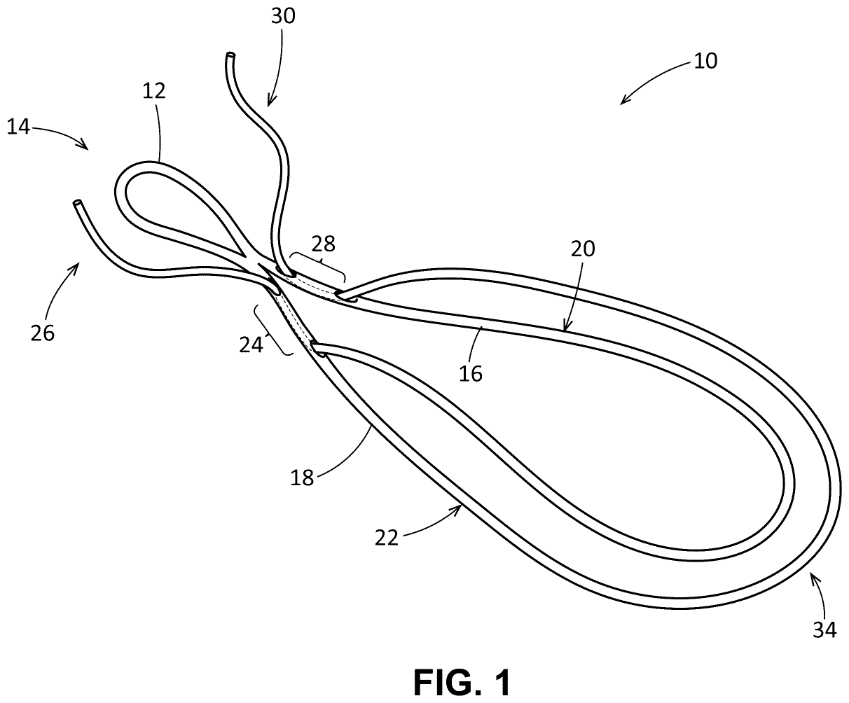

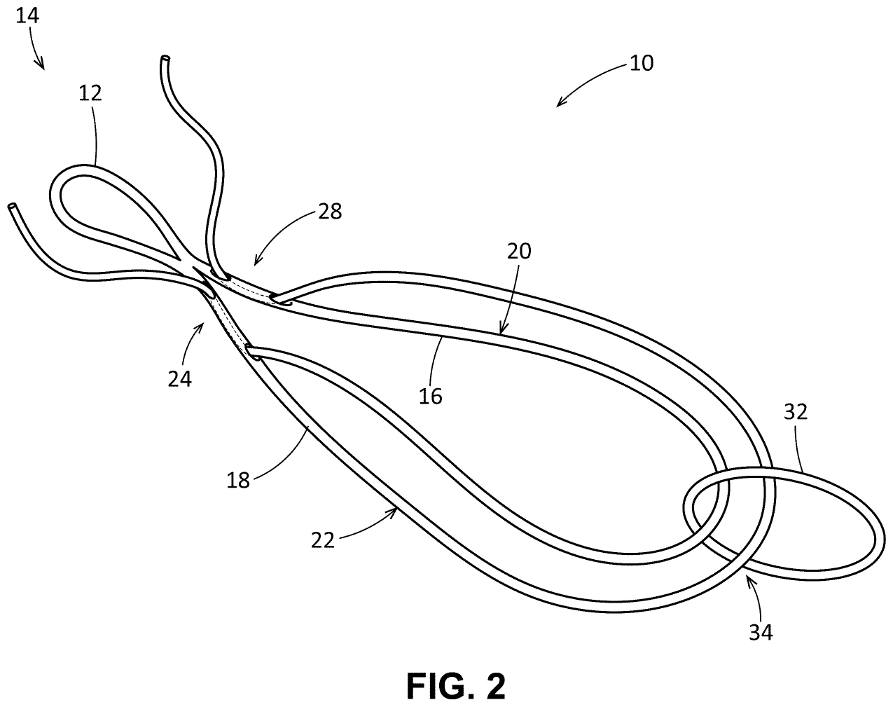

Referring to FIG. 1 and FIG. 2, in some embodiments, a static internal brace device 10 with interwoven adjustable loops includes suture material including a fixed loop 12 at a first end 14 and first and second strands 16, 18 extending from the fixed loop 12 in the direction away from the first end 14. Each strand may include hollow core suture material in some embodiments. In other embodiments, each strand may include any suitable orthopedic suture material, such as but not limited to solid core suture material, suture tape, wire, etc.

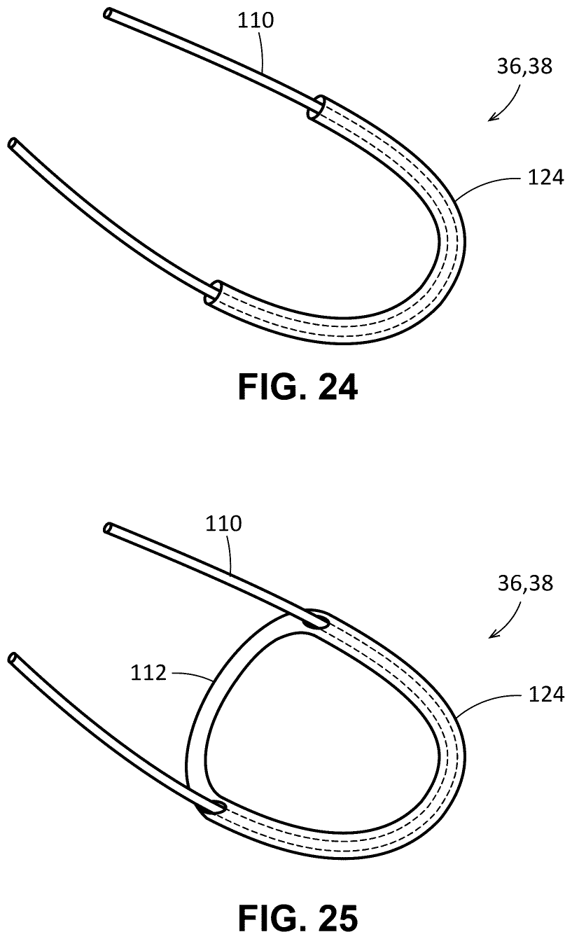

The first strand 16 forms a first adjustable loop 20 and travels through a first self-cinching section 24 in the second strand 18. The self-cinching section 24 can include passing the first strand 16 axially through the hollow core of the second strand 18 in a self-cinching sleeve embodiment. In other embodiments, the self-cinching section includes passing the first strand 16 transversely through the second strand 18, or both transverse passes and axial sleeve passages, as shown in various examples in FIG. 24. The tag end 26 of the first strand 16 exits the first self-cinching section 24 in a direction toward the first end 14, and a user may pull the tag end 26 of the first strand 16 to tighten the first adjustable loop 20.

The second strand 18 forms a second adjustable loop 22 and travels through a second self-cinching section 28 in the first strand 16. The self-cinching section 28 can include passing the second strand 18 axially through the hollow core of the first strand in a self-cinching sleeve embodiment. In other embodiments, the self-cinching section 28 includes passing the second strand 18 transversely through the first strand 16, or both transverse passes and axial sleeve passages, as shown in various examples in FIG. 24. The tag end 30 of the second strand 18 exits the second self-cinching section 28 in a direction toward the first end 14, and a user may pull the tag end 30 of the second strand 18 to tighten the second adjustable loop 22.

As shown in FIG. 1, the fixed loop 12 may be secured to a fixation device, or anchor, such as a suture button, hard anchor, soft button, soft anchor, rigid button, or any other suitable fixation device. Similarly, the second end 34 opposite the first end 14 and fixed loop 12 may be secured to tissue or bone using any suitable fixation device such as a suture button, hard anchor, soft button, soft anchor, rigid button, or any other suitable fixation device.

Referring to FIG. 2, in some embodiments, the internal brace device 10 includes a suture construct with a fixed loop 12 at the first end 14, and first and second suture strands 16, 18 extending from the fixed loop 12 in a direction away from the fixed loop 12. The first and second strands 16, 18 each form an adjustable loop 20, 22 by each passing through a self-cinching section 24, 28 in the opposing strand. A free loop 32 is disposed on the first and second adjustable loops 20, 22 such that the first and second strands 16, 18 pass through the free loop 20 at the second end 34 opposite the first end 14.

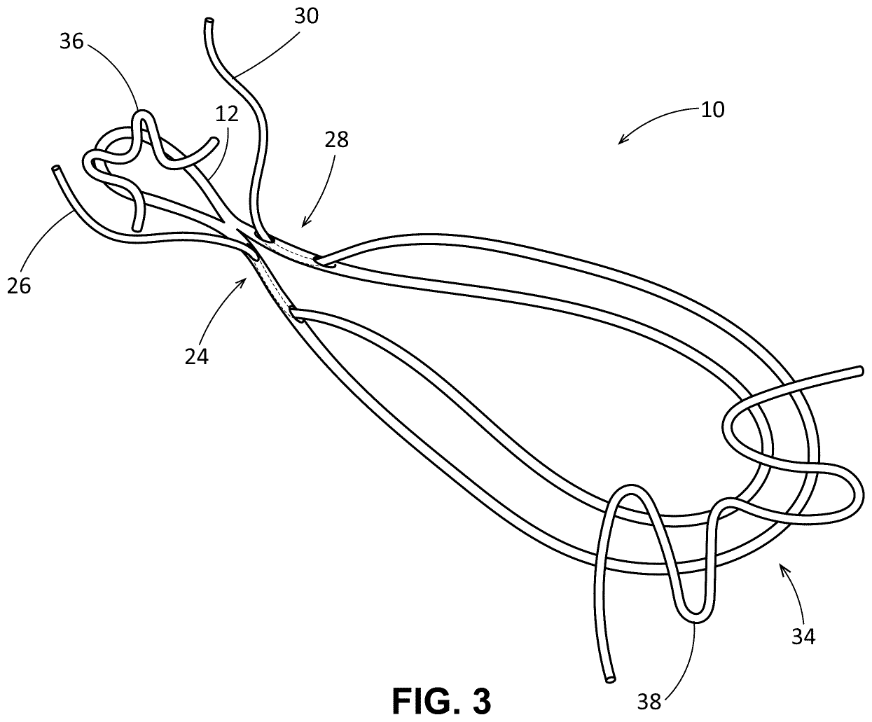

Referring to FIG. 3, in some embodiments, an internal brace device 10 includes a first anchor 36 disposed on the fixed loop 12 and a second anchor 38 disposed on the second end 34. In some embodiments, each anchor 36, 38 may include any suitable anchor material, such as but not limited to flexible suture tape, button, suture material, metal anchor, PEEK anchor, plastic anchor, implant, graft, tissue graft, screw, plate, needle, suture loop, or other orthopedic devices. The first and second anchors 36, 38 are configured to engage opposing bone or tissue structures. For example, in some embodiments, the first anchor 36 is configured to engage the tibia, and the second anchor 38 is configured to engage the femur, such that when the tag ends 26, 30 of the first and second strands 16, 18 are both pulled, the internal brace 10 will tighten across the knee joint and provide a brace across the joint. The first and second self-cinching sections 24, 28 maintain tension on the first and second strands 16, 18 once tightened.

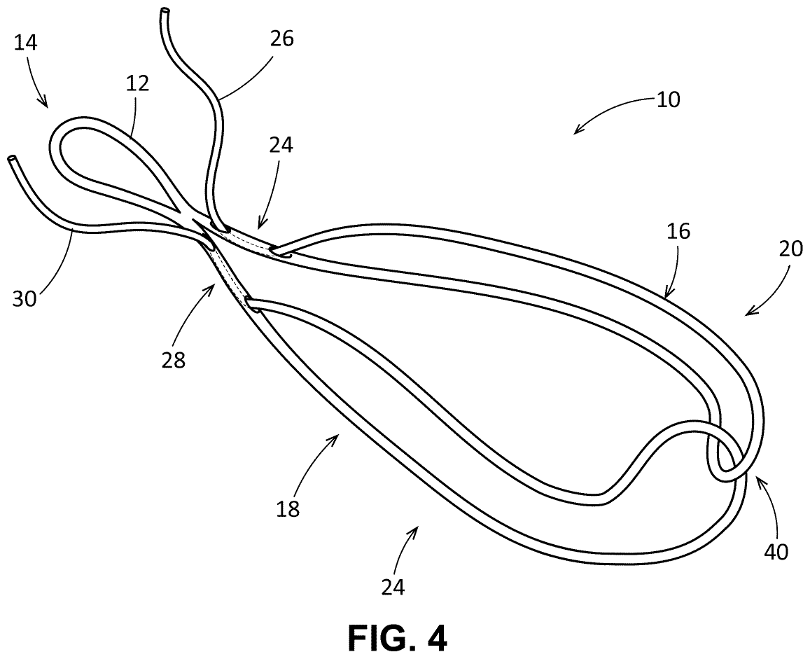

Referring to FIG. 4, an alternative embodiment of an internal brace device 10 includes interlocked adjustable loops 20, 24 with a fixed loop 12 at the first end 14 and first and second strands 16, 18 extending from the fixed loop 12 in a direction away from the first end 14. The first strand 16 interlocks with the second strand 18 and doubles back toward itself, thereby engaging a first self-cinching section 24 near the fixed loop 12 on the first strand 16. The second strand 18 interlocks with the first strand 16 and doubles back toward itself, thereby engaging a second self-cinching section 28 near the fixed loop 12 on the second strand 18. Each self-cinching section 24, 28 may include passing the strand through the hollow core of the strand in a self-cinching sleeve embodiment. In other embodiments, the self-cinching section includes passing the strand transversely through the itself, or both transverse passes and axial sleeve passages, as shown in various examples in FIG. 24.

When tension is pulled against the tag end 26 of the first strand 16, the interlock with the second strand 18 forms a pulley 40 against which tension may be applied. Similarly, when tension is pulled against the tag end 30 of the second strand 18, the interlock with the first strand 16 forms a pulley 40 against which tension may be applied.

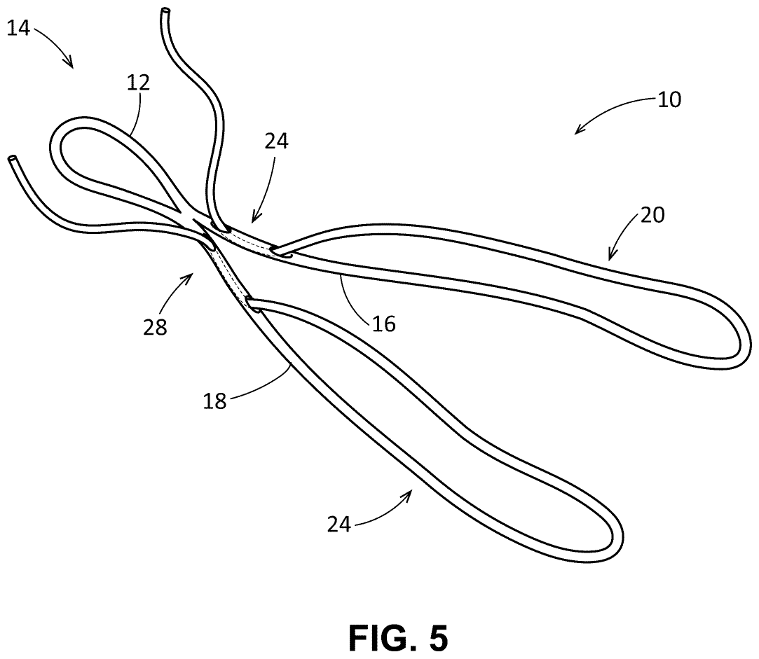

Referring to FIG. 5, an alternative embodiment of an internal brace device 10 includes independent adjustable loops 20, 24. The device includes a fixed loop 12 at a first end 14, and first and second strands 16, 18 extending from the fixed loop 12 in a direction away from the fixed loop 12. The first strand 16 forms a 180-degree adjustable loop 20 and turns back towards itself without crossing the second strand 18. The first strand 16 engages itself near the fixed loop 12 in a first self-cinching section 24. The second strand 18 forms a 180-degree adjustable loop 24 and turns back towards itself without crossing the first strand 16. The second strand 18 engages itself near the fixed loop 12 in a second self-cinching section 28. Each self-cinching section 24, 28 may include passing the strand through the hollow core of the strand in a self-cinching sleeve embodiment. In other embodiments, the self-cinching section includes passing the strand transversely through the itself, or both transverse passes and axial sleeve passages, as shown in various examples in FIG. 24.

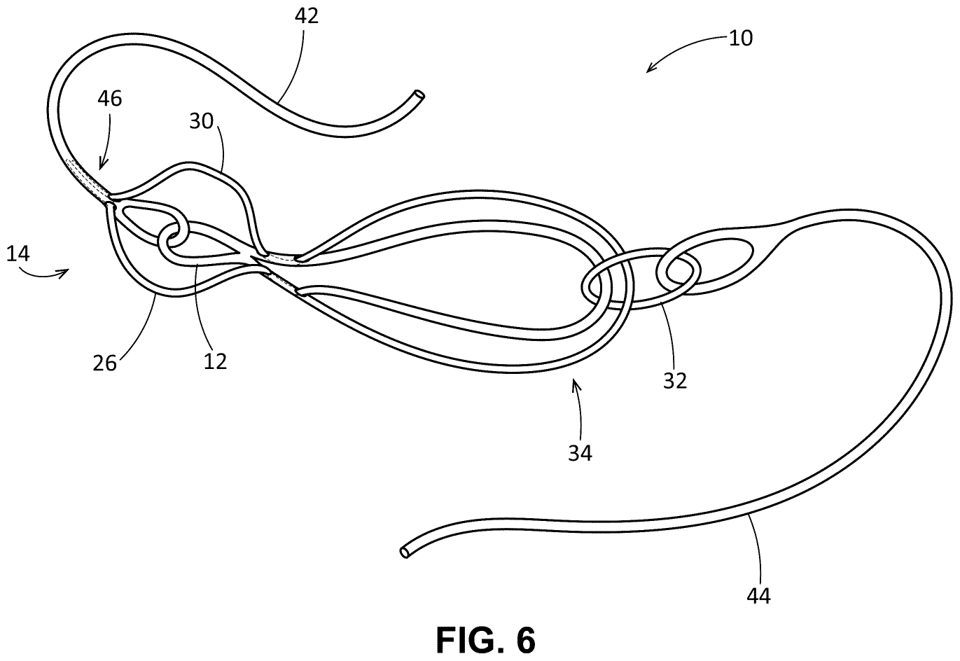

Referring to FIG. 6, an alternative embodiment of an internal brace device 10 includes a first shuttling suture 42 attached to the fixed loop 12 at the first end 14 of the internal brace 10, and a second shuttling suture 44 attached to a free loop 32 at the second end 34 of the internal brace. The first and second tag ends 26, 30 may be disposed axially into one or two tag end sleeves 46 defined in the first shuttling suture 42. By tucking the first and second tag ends 26, 30 of the first and second strands 16, 18 into the tag end sleeve or sleeves 46 of the first shuttling suture 42, the tag ends 26, 30 may be protected during deployment of the device through a tunnel drilled in bone or through tissue. The first and second shuttling sutures 42, 44 facilitate deployment of the internal brace through tunnels drilled in bone on opposite sides of the joint.

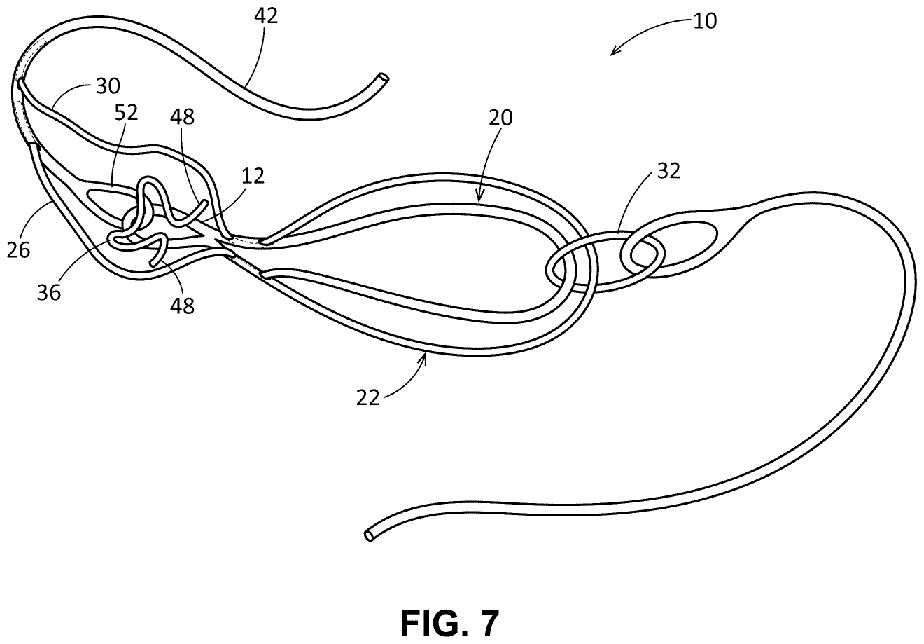

Referring to FIG. 7, an alternative embodiment of an internal brace device 10 includes a self-cinching internal brace device with a free loop 32 attached to first and second adjustable loops 20, 22 and a first anchor 36 on the fixed loop 12. The first anchor 36 in some embodiments includes a soft anchor comprising flexible suture tape with the fixed loop passing transversely through the suture tape, forming a plurality of soft anchor loops on the fixed loop 12. In some embodiments, the tag ends 48 of the soft anchor are configured to extend radially away from the fixed loop 12 on the end of the fixed loop closest to the first and second strands 16, 18 to better facilitate the soft anchor gripping the orifice of a bone tunnel. In some embodiments, the first anchor 36 may include any suitable anchor material, such as but not limited to flexible suture tape, button, suture material, metal anchor, PEEK anchor, plastic anchor, implant, graft, tissue graft, screw, plate, needle, suture loop, or other orthopedic devices. In some embodiments, the first shuttling suture 42 includes a first shuttling suture loop 52 at its proximal end nearest the fixed loop 12, and the first shuttling suture loop 52 extends around both the fixed loop and also the first anchor 36. In further embodiments, the first shuttling suture loop 52 extends between the first anchor 36 and the fixed loop 12.

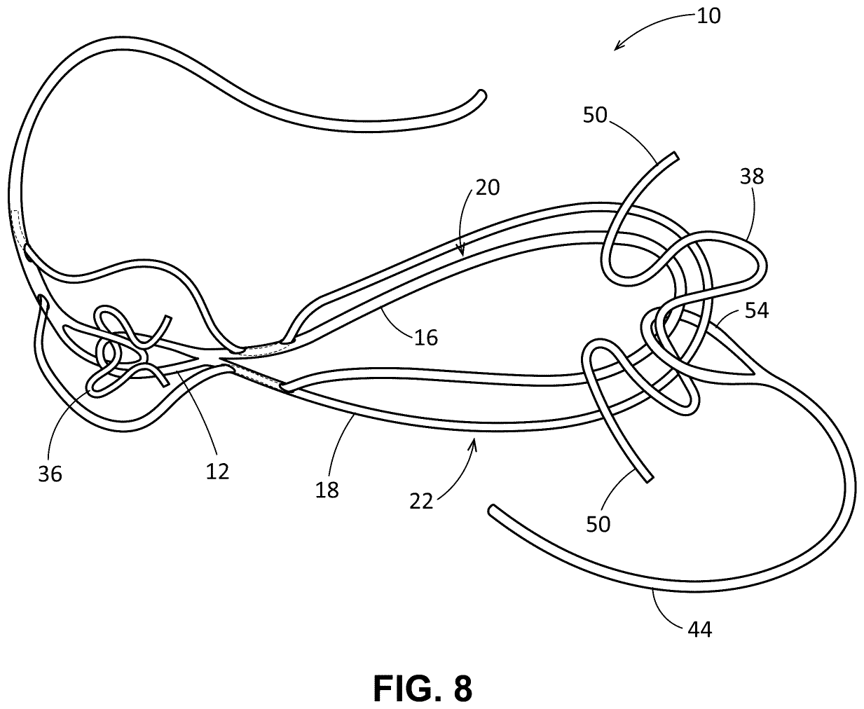

Referring to FIG. 8, an alternative embodiment of an internal brace device 10 includes a self-cinching internal brace with a soft button on both the fixed and adjustable loops at the first and second ends of the brace, respectively. The first anchor 36 includes any suitable anchor, as noted above with respect to FIG. 7. The second anchor 38 also includes any suitable anchor as described herein. In some embodiments, the second anchor 38 includes a soft anchor comprising flexible suture tape, wherein the first and second strands 16, 18 pass transversely in multiple passes through the suture tape material. In some embodiments, the first strand 16 passes through openings in the second anchor 38 separate from the openings in which the second strand 18 passes. In other embodiments, the first and second strands 16, 18 pass through common openings in the second anchor 38 on each transverse pass. In further embodiments, the first and second strands 16, 18 share some openings in transverse passes, but in other passes travel through independent openings in the second anchor 38. Referring further to FIG. 8, in some embodiments, the second anchor 38 comprises a soft button comprising flexible suture tape, and the first and second tag ends 50 of the suture tape material project radially away from the first and second adjustable loops 20, 22 on the end closest to the fixed loop 12, thereby enhancing gripping of the second anchor 38 against an orifice of a bone tunnel. The second shuttling suture 44 includes a fixed second shuttling suture loop 54 in some embodiments. The fixed second shuttling suture loop 54 travels around the first and second adjustable loops 20, 22 and around the second anchor 38. In further embodiments, the fixed second shuttling suture loop 54 travels between the second anchor 38 and the first and second adjustable loops 20, 22.

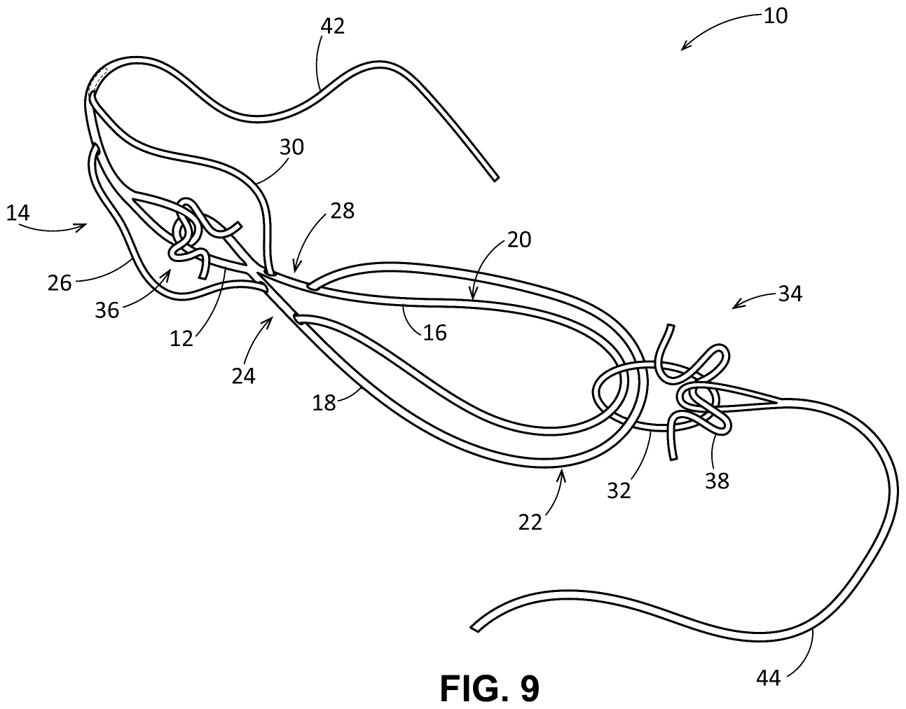

Referring to FIG. 9, an alternative embodiment of an internal brace 10 includes a self-cinching internal brace with first anchor 36 in the form of a soft anchor on the fixed loop 12 and a second anchor 38 in the form of a soft anchor on the free loop 32. The internal brace device includes a fixed loop 12 and first and second strands 16, 18 extending from the fixed loop 12 forming first and second adjustable loops 20, 22 and each passing through a corresponding self-cinching section 24, 28 in the opposing strand. A first anchor 36 in the form of a soft anchor is disposed on the fixed loop 12, and a second soft anchor 38 is disposed on a free loop 32 disposed on the first and second adjustable loops 20, 22 at the second end 34 opposite the first end 14. A first shuttling suture 42 is disposed on the fixed loop 12, and a second shuttling suture 44 is disposed on the free loop 32 which is free to travel on the first and second adjustable loops 20, 22. The second anchor 38 comprises any suitable anchor as discussed herein. By placing the second anchor 38 on the free loop 32, the free loop 32 operates as a pulley against which the first and second adjustable loops 20, 22 may be tightened by pulling the first and second tag ends 26, 30 of the first and second strands 16, 18 through the first and second self-cinching sections 24, 28 in the direction away from the free loop 32.

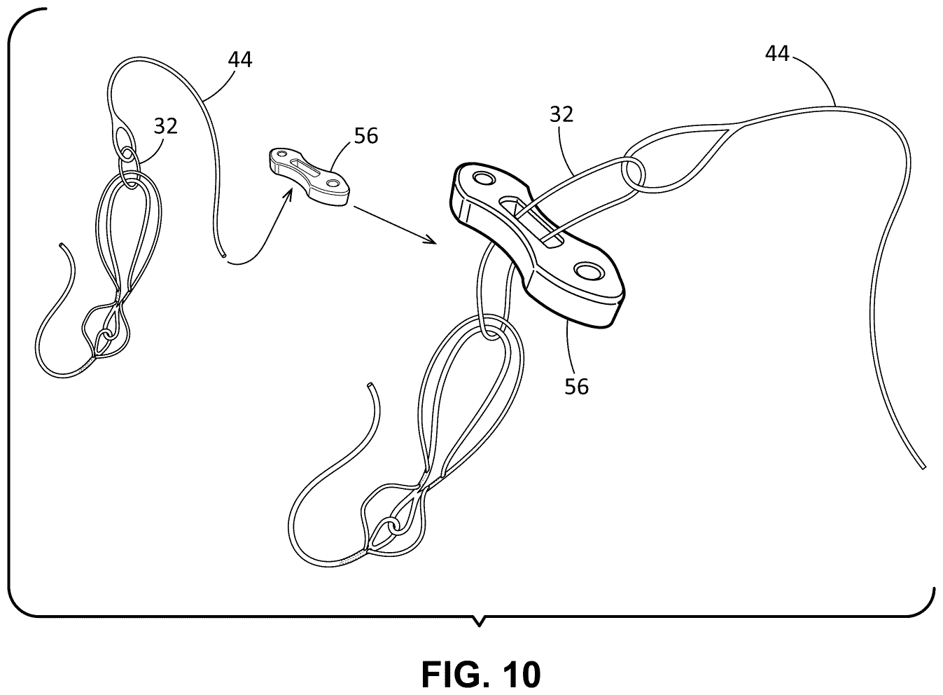

Referring to FIGS. 10-20, a method of installing an internal brace is illustrated in numerous embodiments and steps. As shown in FIG. 10, a first step includes passing the second shuttling suture 44 through an upper fixation device 56 such as a button anchor until the button is disposed on or near the free loop 32. Upper fixation device 56 can take many forms and may be used interchangeably as second anchor 38, shown in FIG. 9. Upper fixation device 56 may be referred to as a femoral fixation device in some embodiments because it is configured to engage the femoral cortex on the upper leg. As shown in FIG. 11, the free loop 32 may looped around one or both ends of the upper fixation device 56 to secure the upper fixation device 56 onto the free loop 32, forming a hitch around the upper fixation device 56. The second shuttling suture 44 may be used to manipulate the free loop 32 around the end of the upper fixation device 56. The shuttling suture 44 may then be removed once the free loop 32 is secure on the button, as shown in FIG. 11.

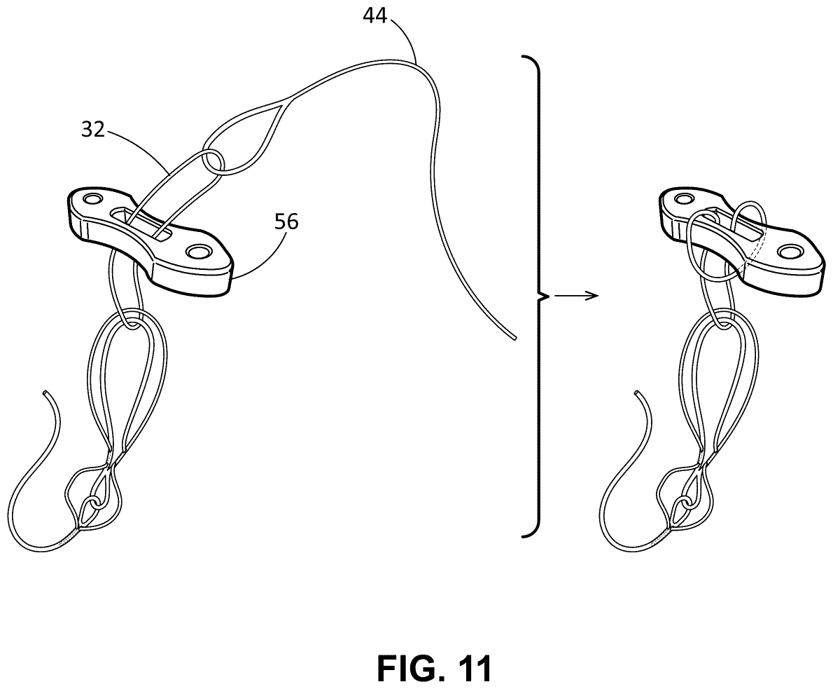

Referring to FIG. 12, an alternative embodiment includes a free loop 32 pre-installed onto the first and second adjustable loops 20, 22 and also pre-installed onto an upper fixation device 56, or button anchor, including first and second holes 58, 60 in the button and a center bridge 62 that cradles the free loop 32. The free loop 32 passes over the bridge 62 and through the first and second holes 58, 60 in the button.

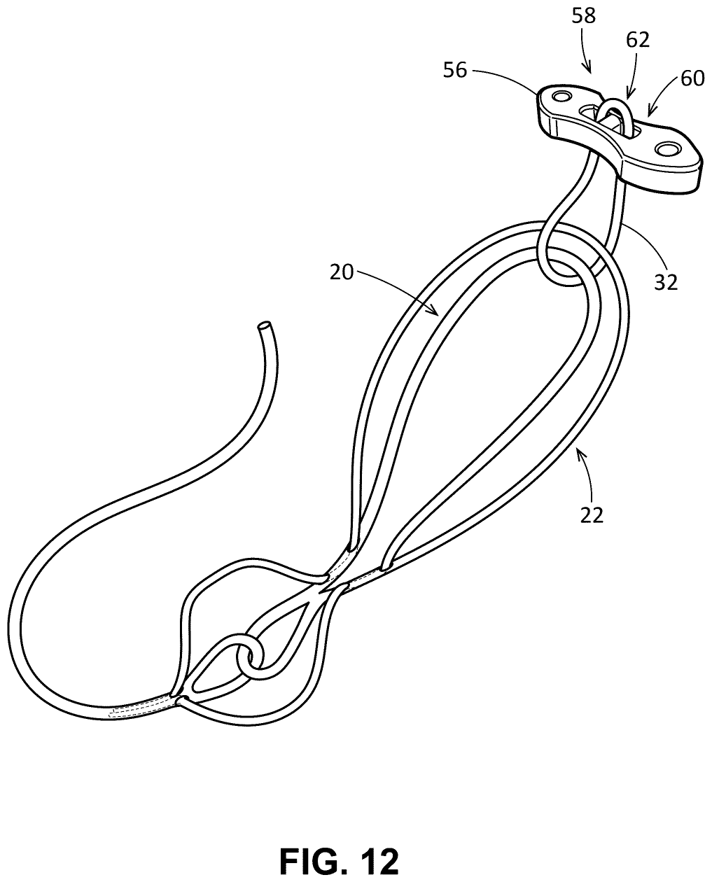

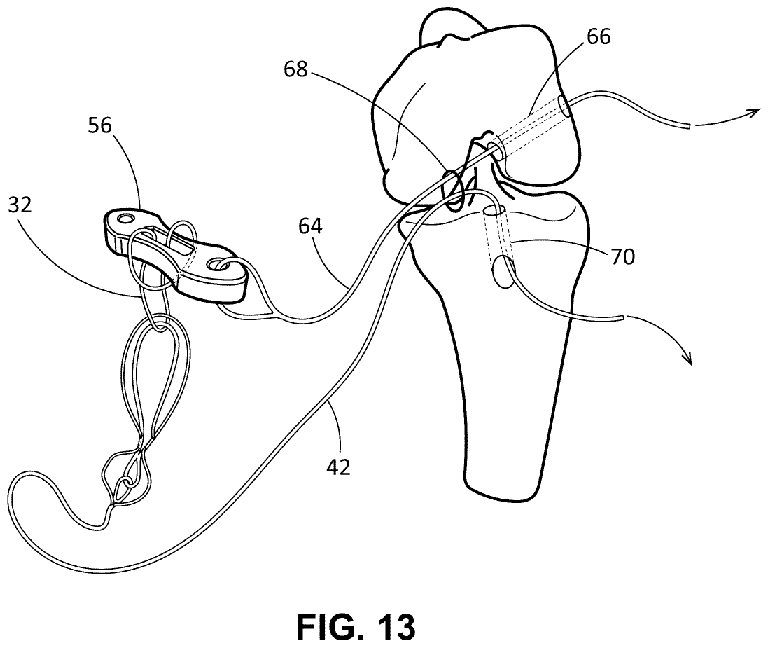

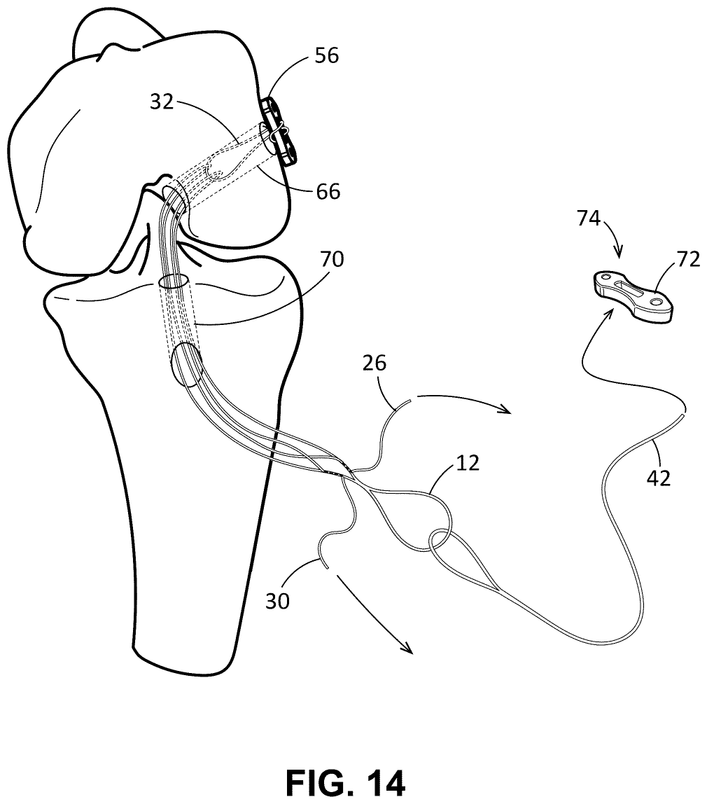

Referring to FIG. 13, a next step includes attaching a button shuttling suture 64 to a lateral end of the upper fixation device 56 such that button is pulled through a tunnel 66 from an end of the button. The button shuttling suture 64 may be pre-installed on an end of the upper fixation device 56. The button shuttling suture 64 is passed through an incision 68 in the knee, such as but not limited to a medial portal incision. The first shuttling suture 42 is then passed through a tunnel drilled in the tibia 70, and the button shuttling suture 64 is passed through a femoral tunnel 66 drilled in the femur. In some embodiments, the button shuttling suture 64 is passed through the femoral side prior to passing the first shuttling suture 42 through the tibial side. Once the upper fixation device 56 is pulled through the femoral tunnel 66, the button will rotate to a square position with the femoral cortex, and the button shuttling suture 64 may be removed from the upper fixation device 56, as shown in FIG. 14. The first shuttling suture 42 attached to the fixed loop 32 is pulled distally, which will seat the upper fixation device 56 against the cortex on the femur on the opposite side of the internal brace. From this position, the first and second tag ends 26, 30 of the first and second strands 16, 18 extend out of the distal end of the tunnel drilled through the tibia 70.

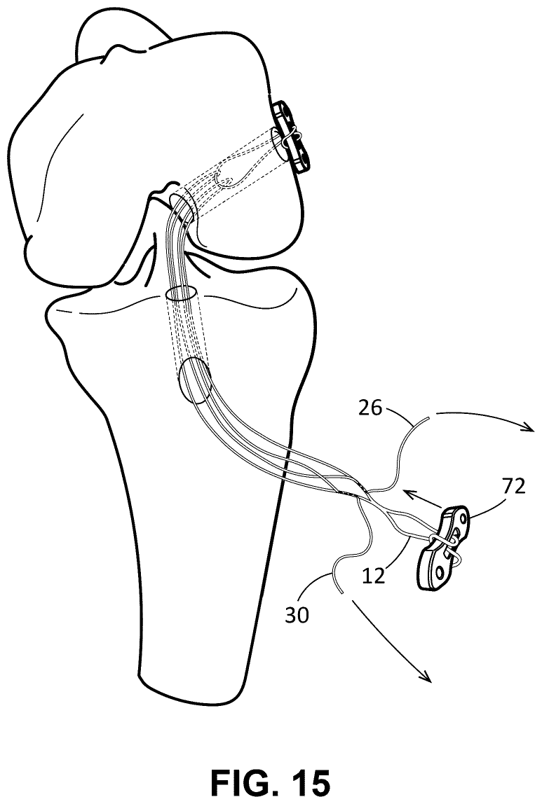

Referring to FIG. 14 and FIG. 15, as a next step, the first shuttling suture 42 attached to the fixed loop 12 is attached to a lower fixation device 72, such as a soft anchor or a button. In some embodiments, lower fixation device 72 is analogous to the first anchor 36 configured for attachment to fixed loop 12. In some embodiments, lower fixation device 72 may be referred to as a tibial fixation device because it is configured to engage the tibal cortex. In some embodiments, the first shuttling suture 42 is installed through the center passage 74 of a second button. The first shuttling suture 42 is then used to manipulate the fixed loop 12 around one or both ends of the second button to secure the fixed loop 12 to the lower fixation device 72, as shown in FIG. 15.

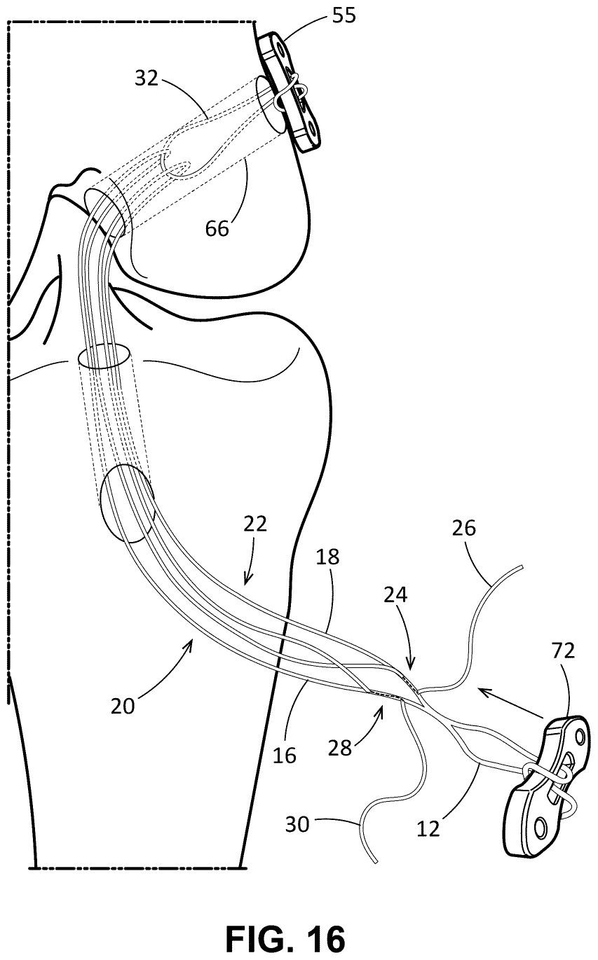

Referring to FIG. 15 and FIG. 16, the first and second tag ends 26, 30 of the first and second strands are then pulled which will shorten the adjustable first and second loops 20, 22 and pull the lower fixation device 72 against the tibial cortex, thereby tightening the first and second self-cinching sections 24, 28 to maintain tension on the internal brace. This allows the internal brace to be tightened in a single stage by pulling the tag ends 26, 30 of the first and second strands 16, 18 in a direction away from the tibia. At this point, the device will function as an internal brace for the ACL. The tag ends 26, 30 of the first and second strands may be trimmed after tightening. From this position, the ligament to be repaired may be fixed to a desired location using a second suture construct attached to the ligament and pulled into the femoral tunnel or into a second tunnel drilled in the femoral side.

As shown in FIG. 16, the upper fixation device 56 on the femoral cortex includes the free loop 32 attached to the upper fixation device 56 and projecting downwardly into the tunnel 66. The length of the free loop 32 extending into the tunnel 66 is less than the tunnel length such that the free loop 32 remains housed within the tunnel in some embodiments. In other embodiments, the free loop 32 is the same length as, or longer than, the femoral tunnel 66 to accommodate operation of the device. The first and second adjustable loops 20, 22 are free to travel over the free loop 32 when tightened, allowing the free loop 32 to operate as a pulley over which the first and second free loops 20, 22 may slide during tightening.

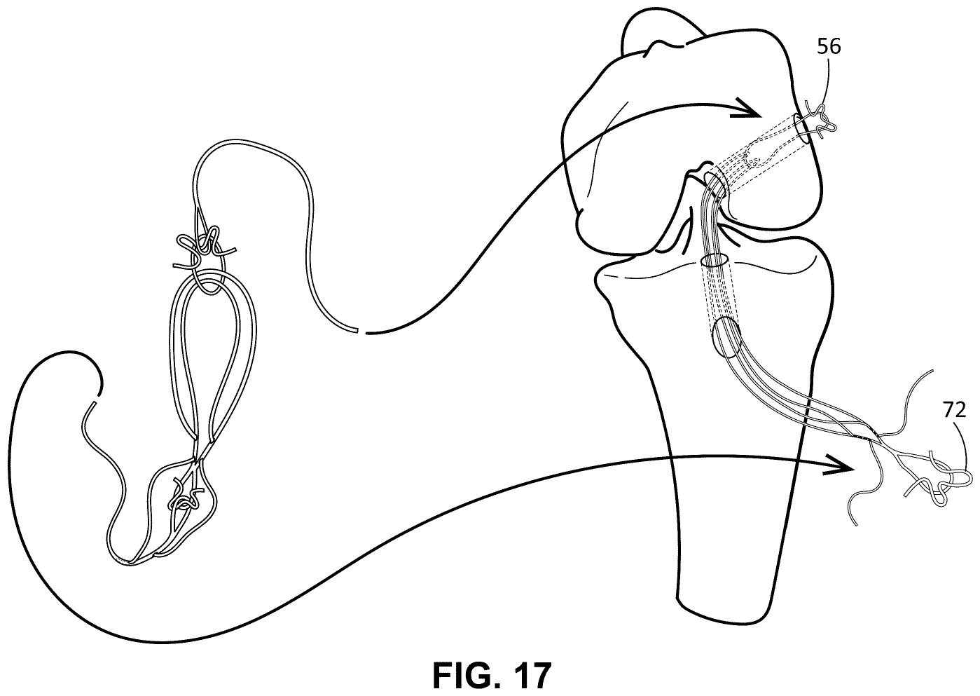

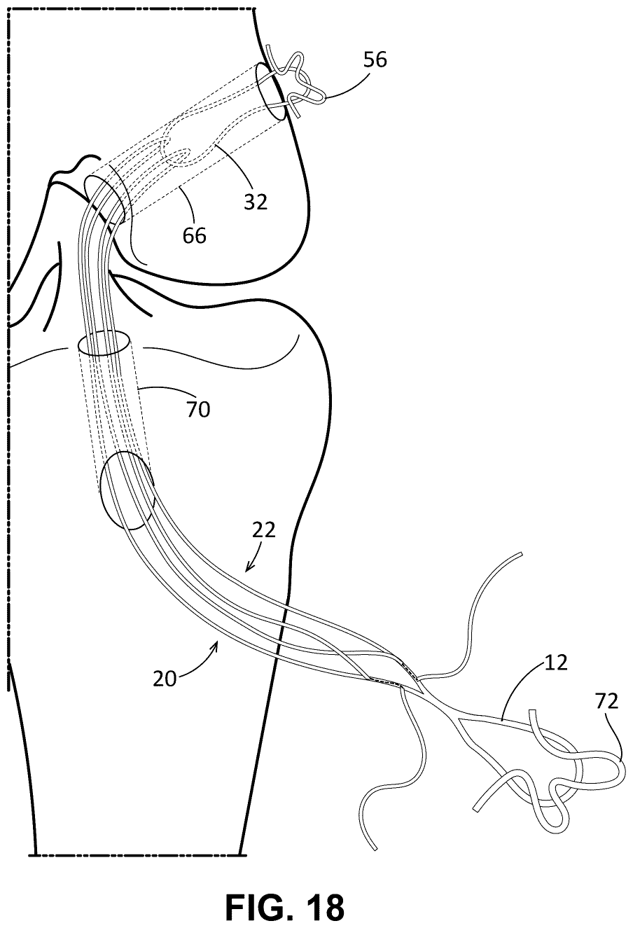

Referring to FIG. 17 and FIG. 18, in an alternative configuration, an upper fixation device 56 the form of a soft anchor, similar to second anchor 38 shown in FIG. 9, is deployed instead of a rigid button on the femoral side. The soft anchor may be attached to the free loop 32, as shown in FIG. 9, or alternatively the soft anchor may be disposed directly on the first and second adjustable loops 20, 22, as shown in FIG. 8. Additionally, as shown in FIG. 17 and FIG. 18, the lower fixation device 72 includes a soft anchor disposed on the fixed loop 12 instead of a rigid button. Lower fixation device 72 is analogous to the first anchor 36 shown in FIG. 9 secured to the fixed loop 12. In other embodiments, a combination of a soft anchor on one end and a rigid anchor on the opposite end may be used. Referring to FIG. 18, in some embodiments, the upper fixation device 56 including a soft button anchor provides fixation of the free loop 32 to the femoral cortex at the orifice of the drilled femoral side tunnel 66, and the soft anchor compresses to form a structure that prevents the soft anchor from sliding into the tunnel when the first and second adjustable loops 20, 22 are tightened. Additionally, as shown in FIG. 17 and FIG. 18, a lower fixation device 72 disposed on the fixed loop 12 includes a soft anchor. The soft anchor of the lower fixation device 72 on the fixed loop 12 provides a similar function as the first soft anchor 36 shown in FIG. 9 to secure the fixed loop 12 at the orifice to the tibial tunnel 70.

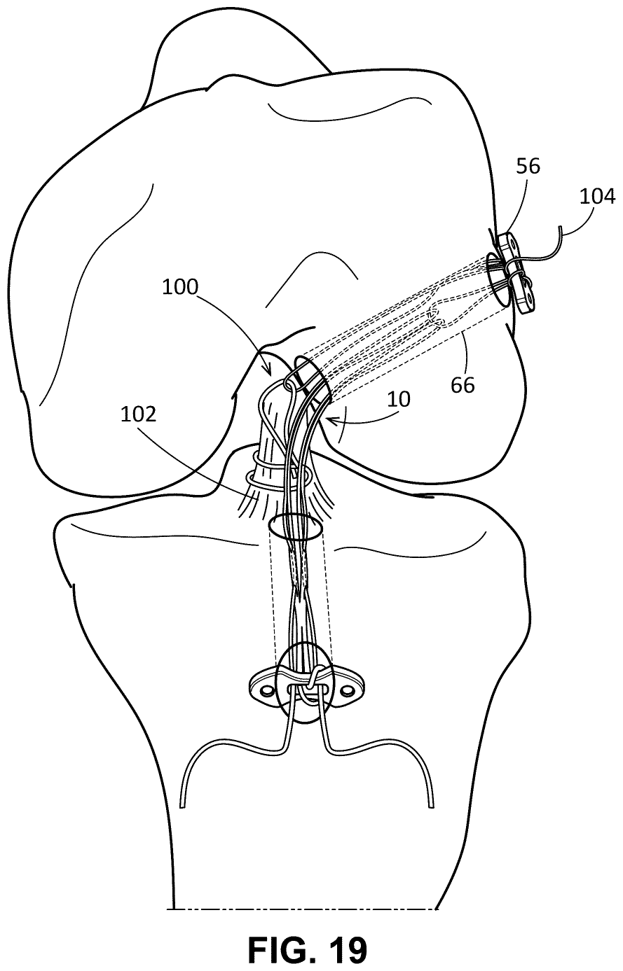

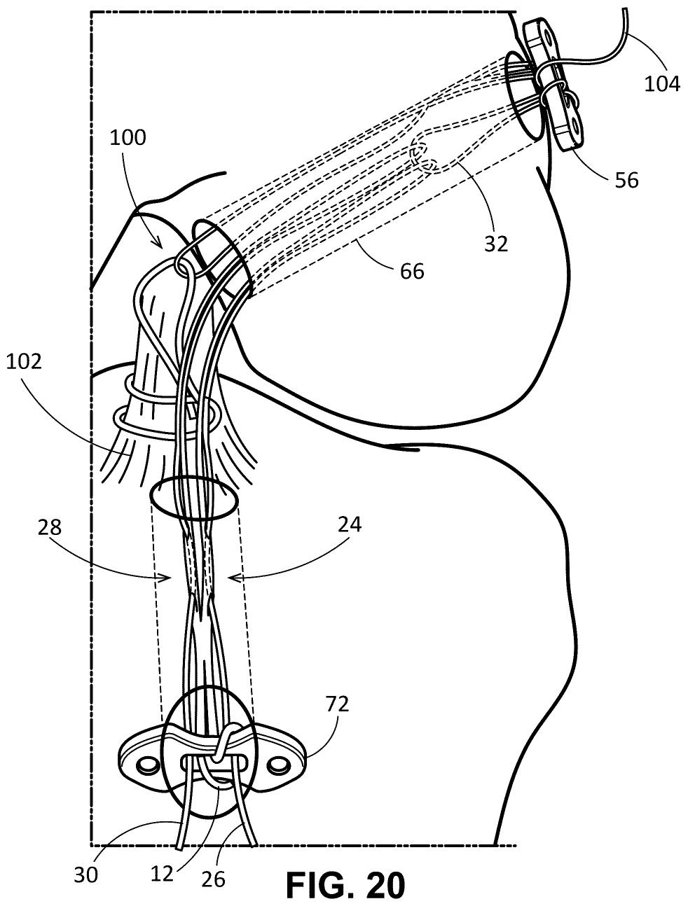

Referring to FIG. 19 and FIG. 20, an internal brace device may be used in combination with a second self-cinching suture construct during an ACL repair. The internal brace 10 is used to span between the tibia and the femur, while the second self-cinching suture construct 100 attaches at one end to the ligament 102 and the other end passes through the femoral tunnel 66, or a second tunnel, toward the upper fixation device 56. The second self-cinching suture construct 100 is attached to the upper fixation device 56 in some embodiments, and the tag end 104 of the second self-cinching suture construct 100 may be pulled to tighten the ligament 102 against the femur in the anatomically correct position while the internal brace 10 maintains tension on the joint. In other embodiments, the second self-cinching suture construct 100 may be attached to a separate anchor on the femoral side.

Referring to FIG. 21A-G, in various embodiments, the self-cinching section on the internal brace or on the second self-cinching suture construct can take many forms. In some embodiments, self-cinching sleeve is formed by passing the suture material into, through, and out of the hollow core section of the suture material, using the hollow core as a self-cinching passage for the suture material to travel through the suture material.

In FIG. 21A, an entirely woven embodiment, the suture strand passes through itself transversely without travelling through the axial length of the hollow core.

In FIG. 21B, a single pass—woven embodiment, the suture travels axially through the hollow core of the suture material, and also includes one or more transverse passes through the suture body, wherein each transverse pass does not travel axially through the hollow core.

In FIG. 21C, a woven—single pass embodiment, the configuration is reversed and the suture travels transversely through itself in one or more passes without travelling axially through the hollow core of the suture, and then enters the hollow core and travels axially through the hollow core a distance.

In FIG. 21D, a woven—single pass—woven embodiment, the self-cinching section includes a first woven section including transverse passes, enters the hollow core and travels axially a distance, and exits the hollow core followed by another series of transverse passes.

In FIG. 21E, a single pass—woven—single pass embodiment, the suture enters the hollow core and travels axially a distance, exits the hollow core and includes a series of transverse passes, and re-enters the hollow core and travels another axial distance before exiting the suture.

In FIG. 21F, a single pass—woven—single pass—woven embodiment, the suture enters the hollow core and travels axially a distance, exits the hollow core and includes one or more transverse passes, re-enters the hollow core and travels axially a distance, exits the hollow core and includes one or more transverse passes.

In FIG. 21G, a multiple single pass embodiment, the suture enters the hollow core and travels axially a distance, exits the hollow core, re-enters the hollow core and travels axially a distance, and exits the hollow core. This pattern may repeat two or more times to achieve the desired level of self-cinching force.

Numerous other combinations of single pass and woven configurations on a single self-cinching section may be achieved by combining single pass and woven sections adjacent to one another.

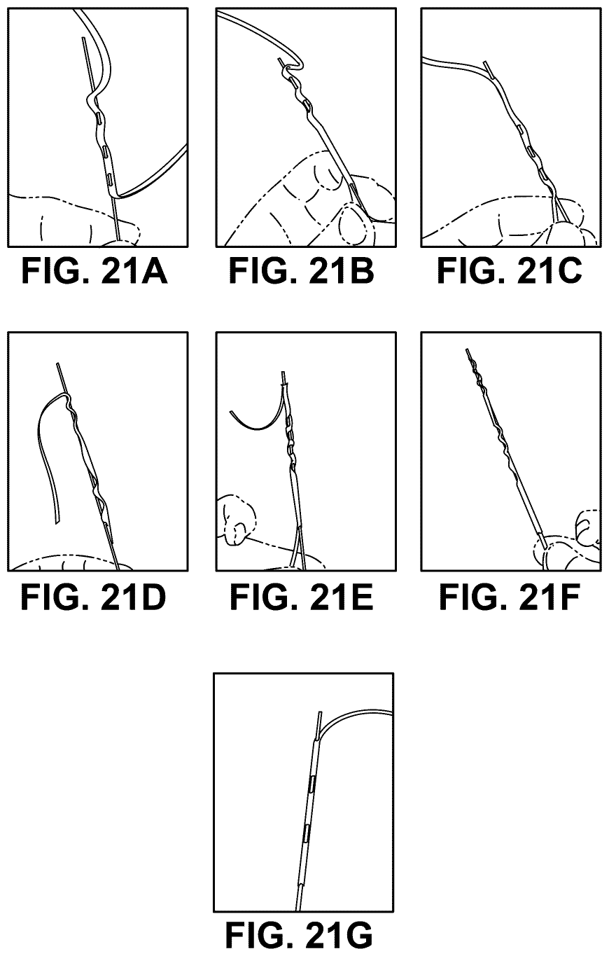

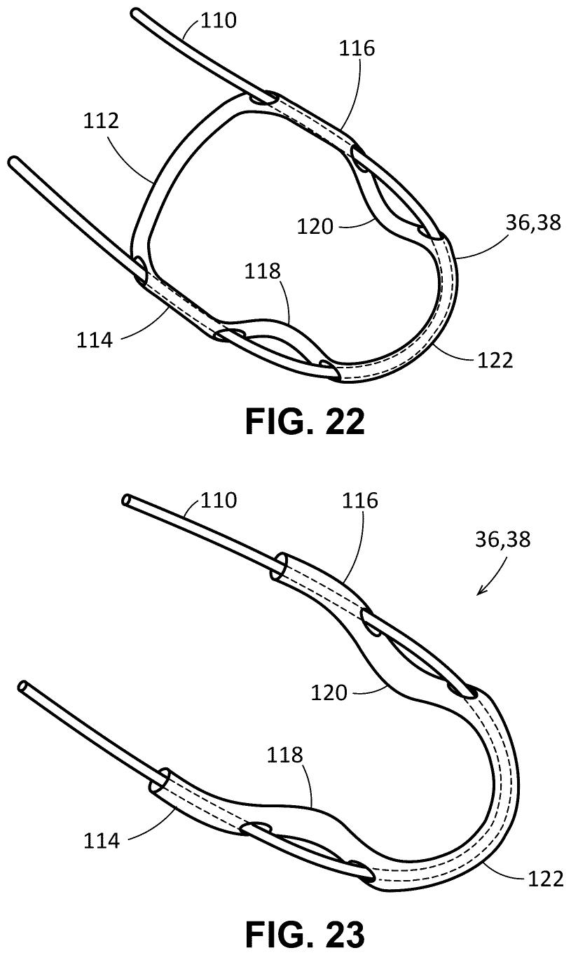

In further embodiments, the first and second anchors may take many forms. In some embodiments, the soft anchor can include a section of flexible surgical tape, wherein the suture material is passed transversely through the suture tape back and forth a number of times. In alternative embodiments, as shown in FIG. 22, the first or second anchor 36, 38 includes a ring-shaped section of hollow core suture material disposed on a suture strand. The first or second anchor 36, 38 forms a bridge 112 spanning across the adjustable loop or the fixed loop, and the suture strand 110 is passed through a first anchor sleeve 114 on one side of the loop, and also through a second anchor sleeve 116 on the opposite side of the loop. This forms a first anchor loop 118 and a second anchor loop 120 between first and second anchor sleeves, respectively, and the distal end of the anchor 122 at the anchor end, which also includes a section of the suture passing through the hollow core of the suture material. As shown in FIG. 23, a similar configuration for the anchor does not include the bridge.

Referring to FIG. 24, an alternative embodiment of the first or second anchor 36, 38 includes a single strand 124 of hollow core suture material disposed on the fixed loop, free loop or the adjustable loop. The suture strand 110 travels through the hollow core of the first or second anchor 36, 38. Another embodiment for first or second anchor 36, 38, shown in FIG. 25, includes a ring-shaped anchor formed from hollow core suture material 124, with the suture strand 110 travelling through a portion of the ring, forming a bridge 112 spanning across the adjustable loop, free loop or the fixed loop.

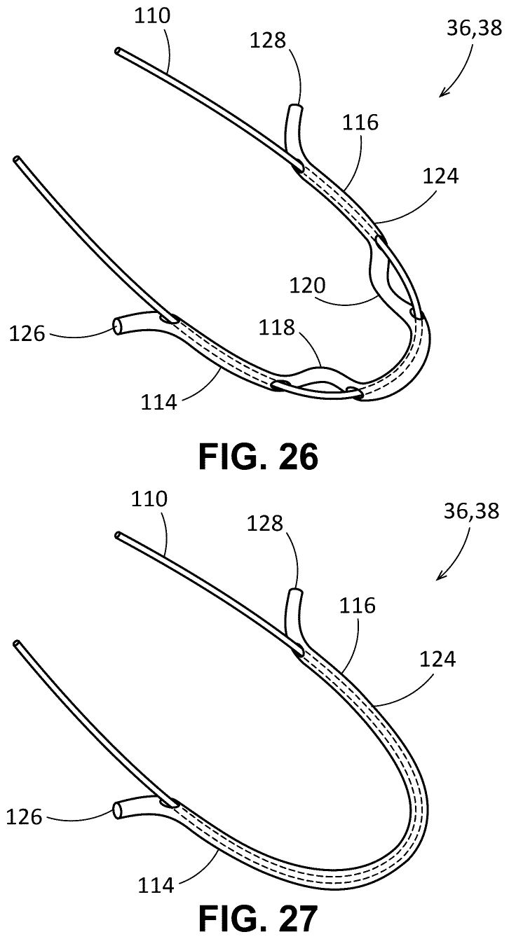

Referring to FIG. 26, an additional embodiment of the first or second anchor 36, 38 includes a single strand 124 of hollow core suture material, where the suture strand 110 passes axially through the hollow core material of the anchor in three passes, forming a first anchor sleeve 114, a second anchor sleeve 116, first anchor loop 118, second anchor loop 120 and first and second tag ends 126, 128 which aid in gathering the anchor against the bone when the adjustable loop of the construct is tightened.

Referring to FIG. 27, an additional embodiment first or second anchor 36, 38 includes a single strand 124 of hollow core suture material, where the suture strand 110 passes axially through the hollow core of the suture material, and the anchor forms first and second tag ends 126, 128 to aid in gathering the anchor against the bone when the adjustable loop of the construct is tightened.

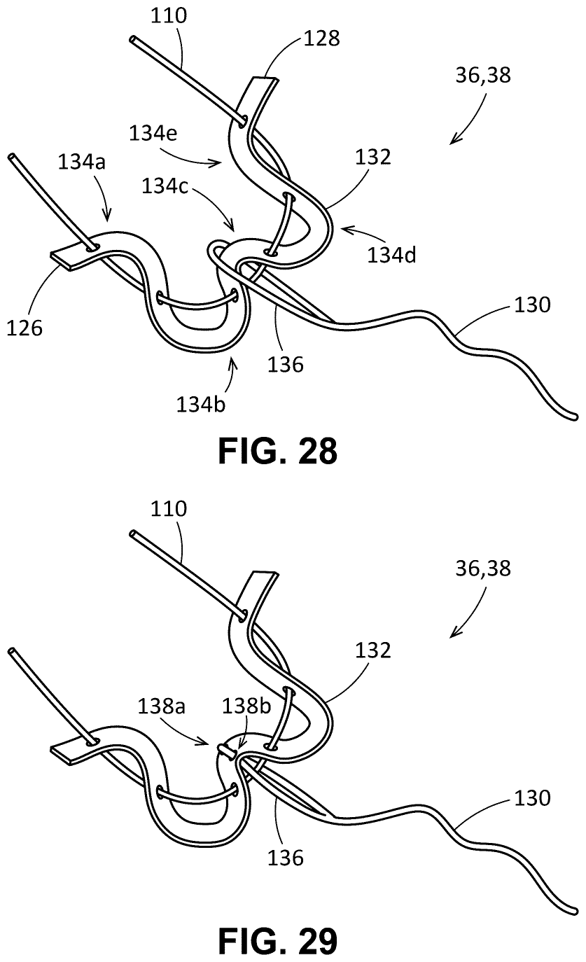

Referring to FIGS. 28 and 29, the first or second anchor 36, 38 may include various embodiments including a shuttling suture 130 to pass the end of the construct through a drilled tunnel or through a drilled and reamed socket in bone. The suture strand 110 includes a soft anchor on the adjustable loop, free loop or fixed loop. The soft anchor includes a section of flexible suture tape 132 in some embodiments. The suture strand 110 passes transversely through the flat side of the suture tape 132 multiple passes to form an anchor. The anchor can thereby gather together when adjustable loop is tightened. In some embodiments, suture strand 110 passes transversely through the anchor six times, forming first and second tag ends 126, 128 and also forming first, second, third, fourth and fifth anchor loops 134a, 134b, 134, 134d, 134e. In some embodiments, a sacrificial shuttling suture 130 includes a shuttling suture loop 136 around both suture strand 110 and anchor material 132. After shuttling the anchor end and anchor through a tunnel or socket, shuttling suture 130 may be cut at shuttling suture loop 136 and removed from the device. In a further embodiment, shown in FIG. 29, the shuttling suture loop 136 passes transversely through first and second anchor holes 138a, 138b to secure the shuttling suture loop 136 to the anchor to better facilitate pulling the anchor and adjustable loop, free loop or fixed loop through a drilled tunnel or drilled and reamed socket in a bone by stabilizing the anchor with the shuttling suture loop 136.

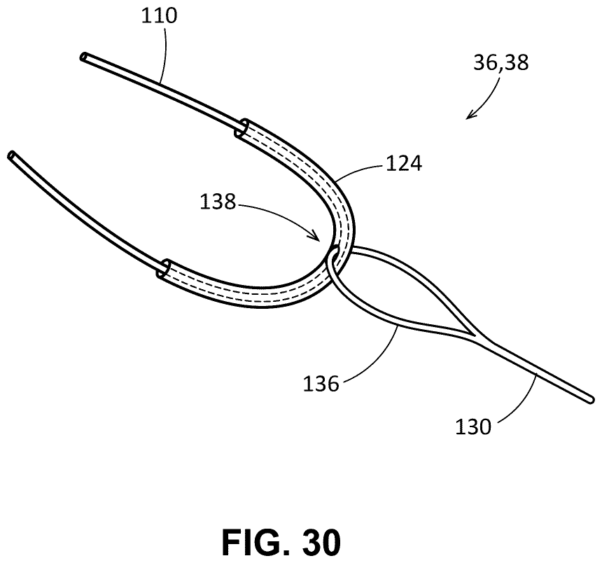

Referring to FIG. 30, in some embodiments, the shuttling suture 130 includes a shuttling suture loop 136 passing transversely through the anchor, when the anchor is in the form of a single section 124 of hollow core suture material disposed on the fixed loop, free loop or adjustable loop. The shuttling suture loop 136 passes transversely through an anchor hole 138 in the anchor material in some embodiments.

Thus, although there have been described particular embodiments of the present invention of a new and useful STATIC INTERNAL BRACE DEVICES AND METHODS, it is not intended that such references to particular embodiments be construed as limitations upon the scope of this invention.

What is claimed is:

1. An internal brace apparatus, comprising:
a suture construct comprising a fixed loop;
a first strand extending in a direction away from the fixed loop; and
a second strand extending in a direction away from the fixed loop,
wherein the first strand passes through a first self-cinching, thereby forming a first adjustable loop, and
wherein the second strand passes through a second self-cinching, thereby forming a second adjustable loop.

2. The apparatus of claim 1, further comprising a first tag end of the first strand extending from the first self-cinching section away from the first adjustable loop.

3. The apparatus of claim 2, further comprising a second tag end of the second strand extending from the second self-cinching section away from the second adjustable loop.

4. The apparatus of claim 3, further comprising a first anchor disposed on the fixed loop.

5. The apparatus of claim 4, wherein the first anchor comprises a soft anchor.

6. The apparatus of claim 5, wherein the first anchor comprises flexible suture tape.

7. The apparatus of claim 4, wherein the second anchor comprises a rigid button.

8. The apparatus of claim 4, further comprising a second anchor disposed on the first and second adjustable loops.

9. The apparatus of claim 8, wherein the second anchor comprises a soft anchor.

10. The apparatus of claim 9, wherein the second anchor comprises flexible suture tape.

11. The apparatus of claim 4, wherein the second anchor comprises a rigid button.

12. The apparatus of claim 3, further comprising a free loop disposed on the first and second adjustable loops.

13. The apparatus of claim 12, further comprising an anchor disposed on the free loop.

14. The apparatus of claim 13, wherein the anchor comprises a soft anchor.

15. The apparatus of claim 14, wherein the anchor comprises flexible surgical tape.

16. The apparatus of claim 3, further comprising a first shuttling suture disposed on the fixed loop and a second shuttling suture disposed on the first and second adjustable loops.

17. The apparatus of claim 3, further comprising:
a free loop disposed on the first and second adjustable loops;
a first shuttling suture disposed on the fixed loop; and
a second shuttling suture disposed on the free loop.

18. The apparatus of claim 17, further comprising a first anchor disposed on the fixed loop and a second anchor disposed on the free loop.

19. The apparatus of claim 18, wherein the first anchor comprises a soft anchor.

20. The apparatus of claim 19, wherein the second anchor comprises a soft anchor.
